(19) United States
(12) Patent Application Publication
(10) Pub. No.: US 2007/0088010 A1
(43) Pub. Date: Apr. 19, 2007

(54) Method of making a single-stage pharmaceutical preparation for oral therapy of dysfunctional uterine bleeding, containing ethinyl estradiol and dienogest

(76) Inventors: Doris Huebler, Uhlstaedt-Kirchhasel (DE); Sabine Fricke, Jena (DE)

Correspondence Address: MICHAEL J. STRIKER, 103 EAST NECK ROAD, HUNTINGTON, NY, 11743, US

(21) Appl. No.: 11/273,426

(22) Filed: Nov. 14, 2005

Related U.S. Application Data
(60) Provisional application No. 60/727,593, filed on Oct. 17, 2005.

(51) Int. Cl. A61K 31/57; A61K 31/56

(52) U.S. Cl. 514/170

(57) ABSTRACT

The single-stage pharmaceutical preparation for oral therapy of dysfunctional uterine bleeding contains at least 21 separately packaged and individual administered daily dosage units in a single container. Each orally administered daily dosage unit contains a combination of ethinyl estradiol and dienogest in low dosage. Administration of this preparation results in a moderate proliferation of the endometrium with complete transformation and slight break-through bleeding in the hormone-free time interval.

### CROSS-REFERENCE

The present invention described hereinbelow is also described in U.S. Provisional Patent Application Ser. No. 601727,593, filed Oct. 17, 2005. Priority of invention based on the aforesaid U.S. Provisional Patent Application is hereby claimed under 35 U.S.C. 119 (e).

### BACKGROUND OF THE INVENTION

The present invention concerns a method of making a single-stage pharmaceutical preparation for oral therapy of dysfunctional uterine bleeding, in which a combination of less than or equal to 0.020 mg ethinyl estradiol and less than or equal to 2 mg of 17α-cyanomethyl-17β-hydroxyestra-4, 9-dien-3-one (dienogest) preferably 0.015 mg ethinyl estradiol and 1.5 mg DNG, is used for at least 21 daily dosage units. The pharmaceutical preparation can also contain seven or less effective-ingredient-free daily dosage units, i.e. daily dosage units, which do not contain any effective ingredients. The number of effective-ingredient-containing daily dosage units determines the number of effective-ingredient-free or no-taking daily dosage units. The total number of both types of daily dosage units or cycle days should be 28. Furthermore the pharmaceutical preparations can also comprise a combination of ethinyl estradiol and dienogest with 90 to 130, preferably 98 to 128, daily dosage units.

Dysfunctional uterine bleeding is a frequent clinical problem in gynecology and is currently treated both with medication and by surgery (M.Y. Bongers, W.J.B. Mol, H.A.M. Broelmann, “Current treatment of dysfunctional uterine bleeding”, Maturitas 2004, 47; pp. 159-174). It is characterized by copious cyclic or acyclic bleeding of greater than or equal to 80 ml and an interval between bleeding of less than or equal to 21 days or of extended bleeding days equal to or greater than 8 days, not however from an organic cause, such as myoma, polyps or cancer. These organic causes must be excluded prior to a positive diagnosis of dysfunctional uterine bleeding.

Dysfunctional uterine bleeding frequently occurs in women during hormonal changes, such as those occurring during puberty and the climacteric stage. Dysfunctional uterine bleeding is caused by insufficient ovarian functioning with no follicular genesis, an absence of Corpus luteum formation with too little activity regarding ovarian estrogen synthesis. Dysfunctional uterine bleeding is frequently connected with anovulation. This sort of anovulation bleeding occurs due to changing estrogen levels. However it can also be caused by chronic anovulation, which is caused by imbalanced estrogen secretion, which releases endometrial proliferation and thus is connected with an increased risk of endometrial cancer. It is known that imbalanced estrogen levels, such as excessive amounts of estrogen or estrogen without balancing counteraction due to gestagen levels, can be the cause of dysfunctional uterine bleeding.

Also taking oral contraceptives can temporarily induce dysfunctional uterine bleeding (very strong and prolonged bleeding) (R. Erny, N. Erny, “Metorrhagia caused by Estrogen-progestin Combinations” in Contracept. Fertil. Sex 1994, 22(2), pp. 93 to 97). Also here the causes are hormonal fluctuations due to forgotten tablets, irregular administration, unsatisfactory pharmico-kinetics or non-optimum estrogen/gestagen ratio.

Numerous therapies have been developed to treat dysfunctional uterine bleeding, in which gestagens alone or an estrogen/gestagen combination are administered in a single-stage (constant or the same daily. dosages in a certain time interval) or multiple stages—two or three stages—(non-constant or different daily dosages of the estrogen/combination, for example estrogen dosages for seven days in a first stage plus estrogen/gestagen daily dosages for fourteen days in the second stage). High hormone dosages are always used in these different therapy schemes with the most different estrogen/gestagen combinations.

I.S. Fraser, in Aust. NZ, U. Obstet. Gynaecol. (1990), 30(4), pp. 353-356, discloses treatment of dysfunctional uterine bleeding by administration of 5 mg Norethisterone, 3 times daily, or 10 mg medroxyprogesterone acetateines, three times daily, as the sole high dosage gestagen for each of fourteen days of the cycle from the 12^{th} to the 25^{th} day.

The treatment of dysfunctional uterine bleeding by high dosage estrogen-gestagen substitution, for example with a single-stage preparation, such as PROSISTON® (0.03 mg ethnyl estradiol/ 6 mg norethisterone acetate) for 20 days, or by administration of a two-stage ovulation inhibiting preparation, such as OVIOL® (0.05 mg ethinyl estradiol—7 daily dosage units—and 0.05 mg ethinyl estradiol/ 0.125 mg desogestrel—15 daily dosage units, is disclosed in “Gynacology”, Kaufmann, Costa and Scharl (editors), Springer Verlag, Berlin, Heidelberg, 2003,139.

R. A. Steiner, D. Fink, “Menstruation Problems”, in Praxis 2002, 91 pp. 1967-1974 discloses likewise that the treatment of dysfunctional uterine bleeding should occur, among other things, with high dosages of gestagens, estrogens or combinations of both. A possible application regimen (see e.g. Steiner) comprises oral administration of 0.01 mg ethinyl estradiol with 2 mg norethisterone acetate for 8 days in decreasing dosages, namely 6, 5, 4, 3, 3, 3, 3, 3 tablets/day.

The treatment of dysfunctional uterine bleeding by a three-stage preparation of ethinyl estradiol (EE)/norgestimate (NGM) followed by a hormone-free placebo administration for three 28 day cycles is demonstrated by A. Davis, A. Godwin, J. Lippman, W. Olsen, M. Kafrissen, in “Triphasic Norgestimate-Ethinyl Estradiol for Treating Dysfunctional Uterine Bleeding”, Obstet. Gynecol. 2000, 96(6), pp. 913-920. The treatment regimen is broken down into a 21-day stage in which the dosage of ethinyl estradiol (EE) is constant (0.035 mg EE) and the dosage of norgestimate (NGM) increases (0.180 mg NGM for seven daily dosage units, 0.215 mg NGM for the next seven daily dosage units and 0.250 mg NGM for the following seven daily dosage units), which is followed by a 7-day hormone-free placebo stage.

U.S. Pat. No. 6,797,282 explains in their specification that generally long duration usage (3 months) of oral contraceptives for treatment of menorrhagia a form of dysfunctional uterine bleeding—can be used for treatment of dysfunctional uterine bleeding.

### SUMMARY OF THE INVENTION

It is an object of the present invention to provide a suitable preparation, which generally reduces dysfunctional uterine bleeding, stops an acute dysfunctional uterine bleeding and preferably prevents repetition of dysfunctional uterine bleeding, which avoids the disadvantages and effects of conventional preparations.

This object and others, which will be made more apparent hereinafter, are attained by a method for making a single-stage pharmaceutical preparation for oral therapy of dysfunctional uterine bleeding.

According to the invention the pharmaceutical preparation comprises at least 21 daily dosage units, each of which contains a combination of less than or equal to 0.020 mg ethinyl estradiol and less than or equal to 2 mg of 17α-cyano-methyl-17β-hydroxyestra 4, 9-dien-3-one (dienogest), preferably 0.015 mg ethinyl estradiol and 1.5 mg dienogest.

This combination is simultaneously used for preparation of a pharmaceutical preparation for oral contraception.

The invention also includes a pharmaceutical preparation containing at least 21 separately packaged and individually administerable daily dosage units, each of which contains a combination of less than or equal to 0.020 mg ethinyl estradiol and less than or equal to 2 mg of 17α-cyano-methyl-17β-hydroxyestra-4, 9-dien-3-one (dienogest), preferably 0.015 mg ethinyl estradiol and 1.5 mg dienogest.

This pharmaceutical preparation can also contain 7 or less effective-ingredient-free daily dosage units. The term “effective-ingredient-free daily dosage units” means daily dosage units, which contain no effective ingredient, however are not no-taking daily dosage units. They are formulated as dosage units administered after the at least 21-day time interval during which the effective-ingredient-containing dosage units are administered, so that the total number of daily dosage units amounts to 28. More particularly, in preferred embodiments the number of daily dosage units each containing a combination of ethinyl estradiol and dienogest amounts to 21, 22, 23, 24 or 25 and the number of effective-ingredient-free daily dosage units amounts to 7, 6, 5, 4 or 3. The number of daily dosage units containing a combination of less than or equal to 0.020 mg ethinyl estradiol and less than or equal to 2 mg of 17α-cyano-methyl-17β-hydroxyestra-4, 9-dien-3-one (dienogest), preferably 0.015 mg ethinyl estradiol and 1.5 mg dienogest, preferably amounts to from 90 to 130, particularly 98 to 128. According to the desires of the women for continuing therapy for dysfunctional uterine bleeding and if necessary for continuing contraception connected with the need for freedom from bleeding over a larger time period, the number of daily dosage units containing a combination of less than or equal to 0.020 mg ethinyl estradiol and less than or equal to 2 mg of 17α-cyanomethyl-17β-hydroxyestra-4, 9-dien-3-one (dienogest), preferably 0.015 mg ethinyl estradiol and 1.5 mg dienogest, preferably amounts to from 365 to 366 with a subsequent administration free time interval.

The single-stage combination preparation used to minimize bleeding during dysfunctional uterine bleeding has a low total steroid dosage in comparison to conventional preparations. It is especially suitable for females during hormone changes, such as puberty and the climacteric period, who, among other things, are interested in a hormonal contraception. Balanced hormone levels, which facilitate a moderate proliferation of the endometrium with a sufficiently complete transformation, are established independently of the number of steroid daily dosage units. A rapid and complete prevention of bleeding from the endometrium requires the administration of the daily dosage units of the steroidal effective ingredients for at least 21 days and effective-ingredient free or administration-free daily dosage units for seven days or less. This stabilizes the cycle and prevents the dysfunctional uterine bleeding. Furthermore continuous hormone administration with 365/366 daily dosage units or 90 to 130 daily dosage units, preferably 98 to 128, daily dosage units with a subsequent administration free-time interval makes an appropriate long bleeding-free time interval possible with simultaneous inhibition of ovulation. The time interval for administration of the pharmaceutical preparation comprises at least three delivery cycles of 28 days, in which the above-described steroid combination is administered for 21, 22, 23, 24 or 25 days and 7, 6, 5, 4 or 3 daily dosage units are effective ingredient free. Also the time interval for administration of the pharmaceutical preparation amounts to at least three cycles of 28 days, in each of which the above-described steroid combination is administered for 21, 22, 23, 24 or 25 days and 7, 6, 5, 4 or 3 daily dosage units are effective ingredient free.

Tests of the Effectiveness of the Claimed Formulations

In a randomized, double-blind placebo controlled clinical study 120 women of ages between 15 and 50 years with dysfunctional uterine bleeding (DUB) symptoms were treated according to a written established protocol for the studies and after exclusion of organic causes of the symptoms with suitable diagnostic procedures (trans-vaginal ultrasound, determination of hormones in the blood). Eighty women received the combination of ethinyl estradiol and dienogest effective ingredients according to the claims below and 40 women received a placebo. The experiment includes a run-in stage of 90 days in which the extent of the bleeding difficulties was evaluated, 40 women had seven treatment cycles of 28 days, 40 women had 2 treatment cycles of 98 days and 40 women had place treatment. All 120 women were observed in a follow-up stage.

The extent or amount of bleeding was determined quantitatively by the alkaline hematin method. For this purpose the women collected the used monthly sanitary device over the entire study and delivered it to the study or testing center. The duration of bleeding and the length of the bleeding-free interval were determined from daily documentation in an electronic notebook or diary.

While the invention has been illustrated and described as embodied in a method of making a single-stage pharmaceutical preparation for oral therapy of dysfunctional uterine bleeding, containing ethinyl estradiol and dienogest, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

I claim:

1. A method for making a single-stage pharmaceutical preparation for oral therapy of dysfunctional uterine bleeding, said method comprising preparing at least 21 daily dosage units, each of which contains a combination of less than or equal to 0.020 mg of ethinyl estradiol and less than or equal to 2 mg of 17α-cyanomethyl-17β-hydroxyestra-4, 9-dien-3-one.

2. The method as defined in claim 1, wherein the combination contains 0.015 mg of said ethinyl estradiol.

3. The method as defined in claim 1, wherein the combination contains 1.5 mg of said 17α-cyanomethyl-17β-hydroxyestra-4, 9-dien-3-one.

4. The method as defined in claim 2, wherein the combination contains 1.5 mg of said 17α-cyanomethyl-17β-hydroxyestra-4, 9-dien-3-one.

5. A method for simultaneously making a contraceptive preparation and a single-stage pharmaceutical preparation for oral therapy of dysfunctional uterine bleeding, said method comprising preparing at least 21 daily dosage units, each of which contains a combination of less than or equal to 0.020 mg of ethinyl estradiol and less than or equal to 2 mg of 17α-cyanomethyl-17β-hydroxyestra-4, 9-dien-3-one for oral contraception and for the oral therapy of the dysfunctional uterine bleeding.

6. A pharmaceutical preparation containing at least 21 separately packaged and individual administered daily dosage units in a single container, wherein each of said daily dosage units contains a combination of less than or equal to 0.020 mg of ethinyl estradiol and less than or equal to 2 mg of 17α-cyanomethyl-17β-hydroxyestra-4, 9-dien-3-one.

7. The pharmaceutical preparation as defined in claim 6, wherein said combination consists of 0.015 mg of said ethinyl estradiol and 1.5 mg of said 17α-cyanomethyl-17β-hydroxyestra-4, 9-dien-3-one.

8. The pharmaceutical preparation as defined in claim 6, wherein the number of the daily dosage units of said combination is 21, 22, 23, 24 or 25, and further comprising 7, 6, 5, 4 or 3 no-taking dosage units, such that a total number of cycle days amounts to 28.

9. The pharmaceutical preparation as defined in claim 6, containing from 90 to 130 of said separately packaged and individual administered daily dosage units in said single container, each of which contains said combination of said ethinyl estradiol and said 17α-cyanomethyl-17β-hydroxyestra-4, 9-dien-3-one.

10. The pharmaceutical preparation as defined in claim 6, containing from 365 to 366 of said separately packaged and individual administered daily dosage units in said single container, each of which contains said combination of said ethinyl estradiol and said 17α-cyanomethyl-17β-hydroxyestra-4, 9-dien-3-one.

11. The pharmaceutical preparation as defined in claim 6, further comprising seven or less effective-ingredient-free daily dosage units, which follow said at least 21 daily dosage units each containing said combination.

12. The pharmaceutical combination as defined in claim 11, wherein the number of said daily dosage units, each containing said combination of said ethinyl estradiol and said 17α-cyanomethyl-17β-hydroxyestra-4, 9-dien-3-one, is 21, 22, 23, 24 or 25 and the number of said effective-ingredient-free daily dosage units is 7, 6, 5, 4 or 3, so that a total number of said daily dosage units amounts to 28.
